(19) United States
(12) Patent Application Publication
(10) Pub. No.: US 2012/0089134 A1
(43) Pub. Date: Apr. 12, 2012

(54) Contactless Photodisruptive Laser assisted Cataract Surgery

(76) Inventors: Christopher Horvath, Desert Hot Springs, CA (US); Vanessa Isabella Vera, Desert Hot Springs, CA (US)

(21) Appl. No.: 12/902,105

(22) Filed: Oct. 11, 2010

(51) Int. Cl. A61B 18/20

(52) U.S. Cl. 606/6

(57) ABSTRACT

Method, apparatus and systems for laser surgery as part of cataract surgery. The implementation thereof includes: A means to perform an anterior or posterior capsulorhexis using a rapid fire sequence of photodisruptive laser pulses, placed to open the capsule for cataract surgery. The system and methods provides the means to target and direct the laser pulse sequence into the desired region of the eye without the need of a patient interface or other contact with the eye.

### BACKGROUND

This application relates to techniques, apparatus and systems for cataract surgery.

Cataract surgery is one of the most common ophthalmic procedures performed. The primary goal of cataract surgery is the removal of the defective lens and replacement with an artificial lens or intraocular lens (IOL) that restores some of the optical properties of the defective lens.

Removing the defective lens requires an opening of the anterior capsule surrounding the lens. This is most commonly done through cutting and tearing a circle shaped opening, using hand tools. This procedure is called capsulorhexis.

Capsulorhexis surgery performed in this manner can involve a high level of skill by the surgeon and can require specialized equipment and supplies, many of which require the assistance of a scrub nurse. The precision in size, centration and continuous edge of the capsulorhexis opening is becoming more and more critical with the advancements of new intraocular lenses (IOL), that require precise placement and symmetrical holding forces from the remaining capsule or bag surrounding the IOL.

Traditional methods for performing a capsulorhexis are based on mechanical cut and peeling techniques.

Another method referred to as YAG laser anterior capsulotomy delivers individual laser pulses with high energy to the eye to assist with the opening of the capsule. The precision and quality of those traditional methods is limited.

More recently, photodisruptive lasers and methods have been introduced that can perform the capsulorhexis opening cut with great precision. However, those methods and systems require a patient interface such as an applanation lens to reference and fixate the eye to the laser system.

Placement of this patient interface adds significant complexity to the surgical setup and can cause undesired or harmful high intraocular pressures levels for the duration of the laser procedure. The patient interface is typically provided sterile and is used only once therefore adding significant cost to the overall cataract procedure.

This invention addresses these limitations by providing a precise photodisruptive based laser capsulorhexis method without the need for a patient interface.

### SUMMARY

This application describes, among others, techniques, apparatus and systems for laser based capsulorhexis surgery. Implementation of the described techniques, apparatus and systems include: determining a surgical target region in the anterior capsule of the eye, and applying laser pulses to photodisrupt a portion of the determined target region to create an opening cut on a capsule of the lens.

The laser pulses are applied to the capsule as an early step of a cataract surgery and before making an incision on the cornea of the eye. The focus of description in this disclosure is an anterior capsulorhexis as always performed for cataract surgery. In some cases, like for example congenital cataract or traumatic cataracts in young patients it is often necessary to also perform a posterior (behind the lens) capsulorhexis. This is typically done after the lens extraction and is considered very challenging to perform with the traditional methods. The here disclosed method and system can equally perform an anterior or posterior capsulorhexis. For ease of description the following disclosure will use the anterior capsulorhexis as an example, but the posterior capsulorhexis shall be considered disclosed as well.

This application describes systems and methods that allow targeted laser pulses to be applied to the eye to make a circular or elliptical incision into the anterior capsule with an adjustable diameter and surgeon defined centration. The surgeon then at a later time can easily peel and remove the piece of the capsule when he enters the eye as part of the cataract procedure.

The here described capsulorhexis procedure is being performed without the use of any patient interface that typically is required to reference and fixate the eye to the laser system. This significantly reduces the surgical complexity, eliminates setup time, reduces the risk for the patient by avoiding transient high intraocular pressures that may be caused by the patient interface through suction and applanation of the cornea and reduces overall surgical cost by not requiring a disposable part.

Instead the laser is applied through mid air without any eye contact to the system and with only manual eye fixation by the surgeon using a hand tool or without any eye fixation at all. The key for the ability to achieve this is the here described method of selecting a fast laser engine, combining it with a specific targeting system and laser scan pattern and thereby achieving a complete laser surgery interaction time of typically only a fraction of 1 second.

Due to the shortness of the laser interaction time and the particular scanning and targeting patterns, great precision and safety can be achieved for the capsulorhexis. Residual movement of the eye during the laser firing will not significantly affect the precision of the cut due to its speed and can be further minimized by manually fixating the eye with the operators hands or a simple tool. Furthermore a fixation light that the patient focuses on can also be used to further immobilize the eye during the laser firing.

The sequence of the here described application includes the following: coarse placement of the patients eye relative to the delivery system exit, setting or confirming the desired cutting diameter and other laser parameters, centering the desired cutting circle relative to the eye, adjusting the depth of the target plane and finally firing the laser which automatically places all laser pulses in a rapid sequence.

Various apparatus and methods are being described in this application that either allow the surgeon to control the centration and depth of the cutting circle by manual movements of parts of the delivery system, or by remote adjustments performed by the surgeon through a user interface or by semi-automatic alignment using tracking devices for the x-y alignment only or finally by a full automatic targeting system using optical tracking such as an iris tracker or other video analysis based tracking for the x-y plane and depth sensing system such as OCT (Optical Coherence Tomography) or video analysis of a converging aiming beam pattern for tracking the z axis. Those semi-automatic (x-y axis only) or full automatic (x-y-z axis) systems will further increase the ease of use and precision of the procedure.

The manual and automatic targeting systems include several aiming laser patterns that allow precise alignment of the laser target area.

In one implementation the laser system is embedded in a slit lamp configuration which allows the capsulorhexis step to be performed outside the sterile field of the operating room in an office setting therefore further minimizing cost and setup. The patient would then be brought into the operating room at a later time to complete the cataract procedure.

In another implementation the system is placed in the operating room and the delivery system can be placed over the patients head.

The placement control of the individual laser pulses during the procedure is automatically controlled by the system in any implementation using scanners and at least one moving lens.

The laser pulses are being applied to the eye in a circular pattern starting posterior to the capsule inside the lens area and then progressively moving anterior in either a slowly rising spiral or in a way that circles are stacked on top of each other, both ways ultimately forming a cylindrical cut zone that starts in the lens area cuts through the capsule and ends in the aqueous humor of the anterior chamber.

The length of the cylinder cut zone allows for misalignment insensitivity before and during the laser firing sequence since the anterior capsule plane that is intended to be cut needs to only fall within the cut cylinder. The middle plane of the cut cylinder is the target plane and is aligned to coincide with the capsule plane intended for cutting. The actual cutting of the capsule will happen with only a few circles or spirals within the entire cut cylinder. With the here proposed preferred range of laser repetition rate, spot separation and cut diameter, those few circles or spirals will be typically cut in a time frame <100 ms therefore not allowing any remaining eye movement to significantly distort the cutting precision.

### BRIEF DESCRIPTION OF FIGURES

FIG. 1 illustrates an overview of an eye.

FIG. 2 illustrates a structure of a lens of an eye with the surrounding capsule and an intended opening plane for the capsule.

FIG. 3 illustrate a structure of a lens of an eye with the surrounding capsule and an intended opening circle for the capsule in top view.

FIG. 4 illustrates the steps of a photodisruptive treatment of the capsule and spreading of the bubbles along a circle.

FIG. 5 illustrate the scanning pattern of a photodisruptive procedure cutting through the capsule in a sequence of circles arranged to form an upward cylinder.

FIG. 6 illustrate the scanning pattern of a photodisruptive procedure cutting through the capsule in a upward spiral.

FIG. 7 illustrate a top view of the lens and capsule with a pattern of aiming laser spots focused on the intended cutting circle.

FIG. 8 illustrate a side view of the lens and capsule with several converging aiming laser beams being focused on the intended cutting circle.

FIG. 9 illustrate a side view of the lens and capsule with a single converging aiming laser beams being focused and continuously scanned around the intended cutting circle.

FIG. 10 shows the functional blocks of the surgical system, where the delivery system position relative to the eye is manually controlled by the surgeon.

FIG. 10b illustrates the manual system and method procedure sequence.

FIG. 11 shows the functional blocks of the surgical system, where the delivery system position relative to the eye, the scanning system and the laser engines are automatically adjusted and controlled through the feedback of a tracking (semi-automatic system) and also a depth sensing device (full automatic system).

FIG. 11b illustrates the semi-automatics system and method procedure sequence.

FIG. 11c illustrates the full-automatics system and method procedure sequence.

### DETAILED DESCRIPTION

Preferred embodiments of the invention are illustrated in the figures.

This disclosure describes, among others, techniques, apparatus and systems for photodisruptive laser based capsulorhexis procedure. Implementation of the described techniques, apparatus and systems include: determining a surgical target region in the anterior capsule of the eye, and applying laser pulses to photodisrupt a portion of the determined target region to create an opening cut on a capsule of the lens.

The here disclosed method and system can equally perform an anterior or posterior capsulorhexis. For ease of description the following disclosure will use the anterior capsulorhexis as an example, but the posterior capsulorhexis shall be considered disclosed as well.

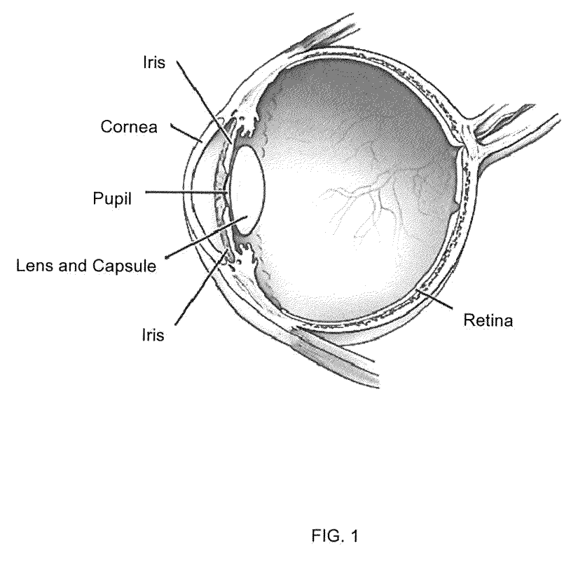

FIG. 1 illustrates the anatomy of a human eye including the cornea, the anterior chamber, the iris, the capsule and the lens inside the capsule.

When the lens develops a cataract it becomes cloudy and at some point cataract surgery might be performed to remove the lens and often replace it with an artificial intra ocular lens (IOL). In order to access the lens a hole must be created in the front (or back) part of the capsule that surrounds the lens. This part of the procedure is referred to as anterior (or posterior) capsulotomy or circular continuous capsulorhexis, depending on the method or technique used to open the capsule (Cataract Surgery: Technique, Complications, and Management, Roger Steinert, Saunders; 2 edition, 2003).

As described in (Kurtz et al., US Patent Application: Pub. No.: US 20090171327) the capsulerexis part of the cataract surgery relies currently on crude laser or manual methods that offer only limited precision and repeatability.

More recently, photodisruptive lasers and methods have been introduced that can perform the capsulorhexis incision with great precision. For example (Kurtz et al., US Patent Application Pub, No.: US 20090149840). However, those methods and systems require a patient interface such as an applanation lens to reference and fixate the eye to the laser system for example (Juhasz at al., U.S. Pat. No. 6,254,595), (Kurtz et al., US Patent Application: Pub. No.: US 20090131921) or (Lummis at al., US Patent Application Pub. No US 20080071254).

This application describes systems and methods that allow targeted laser pulses to be applied to the eye to make a circular or elliptical incision into the anterior capsule with an adjustable diameter and surgeon defined centration without the use of any patient interface. The surgeon then at a later time can easily peel and remove the piece of the capsule when he enters the eye as part of the cataract procedure.

The laser pulses are applied to the capsule in a non sterile office setting or in the operating room as an early step of a cataract surgery and before making an incision on the cornea of the eye.

There are several advantages of performing this here described photodisruptive laser capsulorhexis procedure without the use of any patient interface that typically is required to reference and fixate the eye to the laser system. The lack of a patient interface significantly reduces the surgical complexity and setup time since a patient interface requires precision docking and involves some suction activation typically around the outside of the limbus to stabilize and fixate the eye relative to the delivery system of the laser system.

The lack of a patient interface also eliminates the risk of transient high intraocular pressures (IOP) for the patient that may be caused by the patient interface through suction and applanation of the cornea. Transient IOP values of over 65 mm of Mercury and sometimes over 100 mm of Mercury during the applanation and suction phase of LASIK procedures have been reported (Arturo Chayet, “How IOP Affects LASIK Outcomes”, Ophthalmology Management, 212001) or (Haixia Zhao et al., “Research on Influences of Transient High IOP during LASIK on Retinal Functions and Ultrastructure”, Journal of Ophthalmology, Volume 2009, Article ID 230528).

These transient high IOP levels are particularly concerning for cataract patients that are also affected by Glaucoma since they usually have a damage in the optic nerve or retinal nerve fiber layer loss due to previously elevated IOP (S. Goyal, “Refractive Surgery: A Glaucoma Specialist's Perspective” Cataract & Refractive Surgery Today Europe I January 2010).

Another advantage of performing the laser capsulorhexis procedure without a patient interface is the reduction of overall surgical cost since the patient interface is typically provided sterile and disposable and therefore only used once.

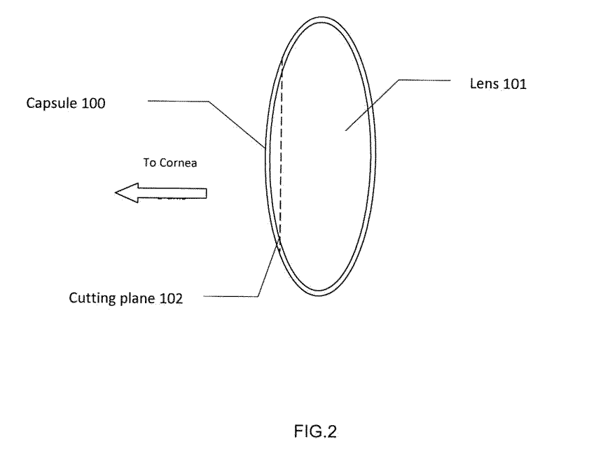

FIG. 2 illustrates the capsule 100 and the lens 101 in a more detailed side view. The lens is typically 6-10 mm in diameter and has a thickness (z-axis) of 2-4 mm. The capsule bag around it typically has a thickness of 20 microns only. The dotted line 102 in FIG. 2 indicates a typical desired cutting plane to achieve a typically circular opening in the anterior capsule at a diameter of 3-8 mm centered on the main optical axis of the lens.

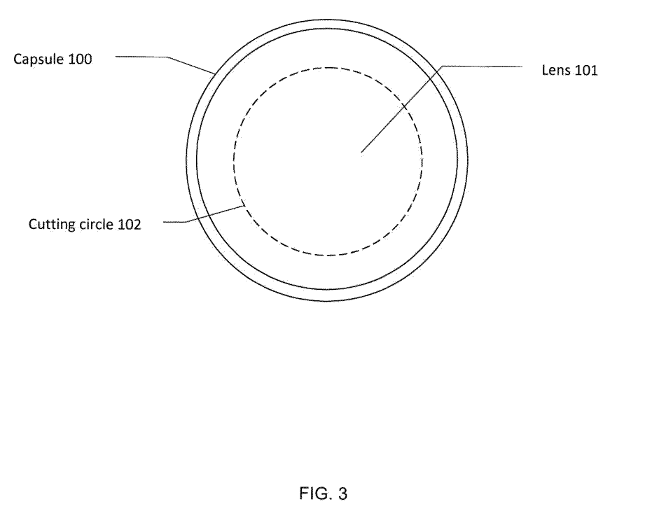

FIG. 3 shows the same lens from a front view with the intended cutting circle 102 centered on the main axis of the lens. The iris that even in a fully dilated stage would typically partially overlap the lens on the outside is here omitted.

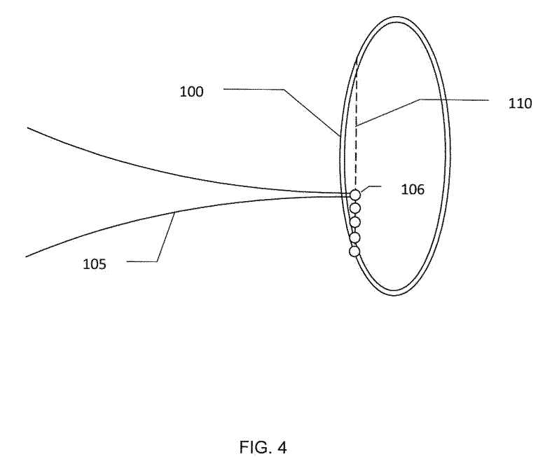

FIG. 4 illustrates the photodisruptive laser pulses being focused on the intended cutting plane and being scanned in a typically circular sequence around the optical axis of the lens. These laser pulses are applied through mid air without any eye contact to the system and with only manual eye fixation by the surgeon using a hand tool or without any eye fixation at all. The individual laser pulses deliver a beam of high peak power onto a small spot size for a ultra short time period onto the target tissue within the eye. This laser tissue interaction has been well characterized and is being used in numerous surgical systems, for example in all-laser LASIK surgery.

As described in (Kurtz et al., US Patent Application: Pub. No.: US 2009/0171327), through this laser-induced lens fragmentation process, laser pulses ionize a portion of the molecules in the target region. This may lead to an avalanche of secondary ionization processes above a “plasma threshold”. These concentrated energy pulses may gasify the ionized region, leading to the formation of cavitation bubbles. These bubbles may form with a diameter of a few microns and expand with supersonic speeds to 50-100 microns. As the expansion of the bubbles decelerates to subsonic speeds, they may induce shockwaves in the surrounding tissue, causing secondary disruption.

Both the bubbles themselves and the induced shockwaves carry out the goal of the procedure: the cutting of the targeted capsule region 102.

The key for the ability to achieve this cutting without any patient interface is the here described method and system of selecting a high repetition rate laser engine 200 in the range of 10 kHz to 10 MHZ and combining it with a specific targeting system and laser scan pattern.

The optical delivery system 220 is configured to scan 230 the laser pulses with a pulse energy in the range of approximately 0.5 microJ to 50 microJ, a separation of adjacent target areas in the range of approximately 1 micron to 30 microns and a pulse duration in the range of approximately 0.005 picoseconds to 50 picoseconds.

A typical capsule opening cutting circle as illustrated in FIG. 4 with a diameter of 5 mm, performed by a typical ultrashort pulsed laser firing at a repetition rate of 200 kHz and a typical spot separation of 10 microns will be completed in about 8 ms.

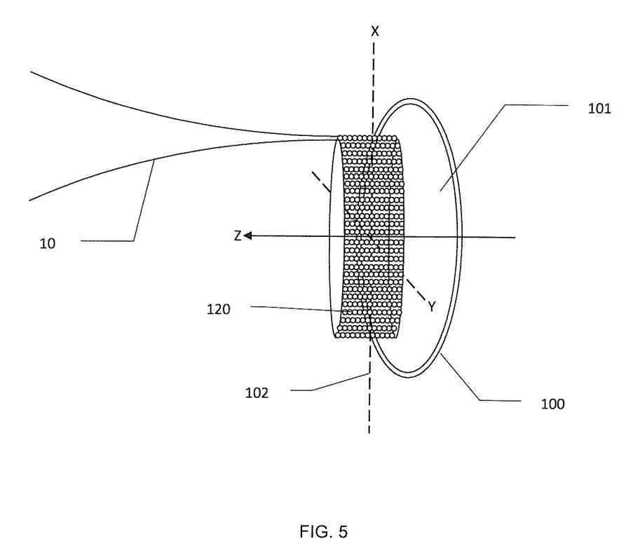

In a scanning pattern as illustrated in FIG. 5 where multiple of these cutting circles are placed on top of each other starting typically 1 mm posterior and ending 1 mm anterior to the capsule and where each successive circle is placed typically 20 microns anterior to the last circle (z-axis moving upwards) the entire cutting cylinder 120 will consist of 100 circles. This entire cutting cylinder will be therefore completed in under 1 second (about 800 ms).

Smaller cutting cylinder margins (length in z-axis) down to +/−0.1 mm can be achieved through surgeon experience and automatic tracking devices as described further down.

I another embodiment a low energy high repetition rate oscillator based laser system can be used to perform the desired cutting. Typical repetition rates of >1 MHz allow for even faster cutting of the desired pattern (L. Goldberg, Ophthalmology Management, “The Femto LDV: A Low Energy Laser Delivery System”, January 2008).

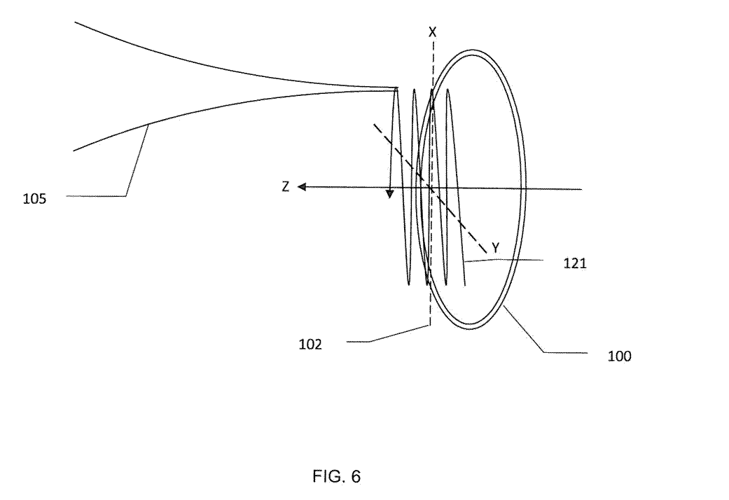

A very similar cutting cylinder can be achieved by scanning the laser in an upward spiral 121 (from posterior to anterior of the capsule) as illustrated in FIG. 6.

This typical combination of parameters allows for a combined misalignment in the z axis of +/−1 mm. Any laser tissue reaction below the capsule (inside the lens) and above the capsule (anterior chamber filled with aqueous humor) is considered no impact and no risk, since the lens will be removed in the following cataract surgery and the aqueous humor is a liquid similar to water and will absorb the laser pulse and cavitation bubbles without any lasting effect.

The only criteria for a successful cut of the anterior capsule is for the intended target plane to fall somewhere within this example of a 2 mm high (z-axis) cutting cylinder. Typical combined alignment errors are typically below 2 mm in the z axis and therefore an even shorter cutting time is easy achievable.

The combined misalignment that needs to be considered consists of an initial depth (z-axis) calibration misalignment of the delivery system, a tilt mismatch between the desired cutting plane and the laser focal plane throughout one cutting circle and any eye movement in the z-axis during the procedure time.

All 3 sources of potential misalignment in the z-axis can be considered well controlled within the large margin of +/−1 mm due to the short time of laser-tissue interaction.

This typical selection of laser firing and scanning parameters achieves a complete laser-eye surgery interaction time of typically less than 1 second. Residual movement of the non fixated eye during the laser firing will not significantly affect the precision of the cut due to its speed and can be further minimized by manually fixating the eye with the operator's hands or a simple tool. Furthermore a fixation light that the patient focuses on can also be used to further immobilize the eye during the laser firing.

The alignment of the delivery system relative to the target area of the eye can be broken down into a lateral alignment (x-y-axis) which is perpendicular to the main optical axis of the eye and a depth alignment (z-axis) which is along the main optical axis of the eye.

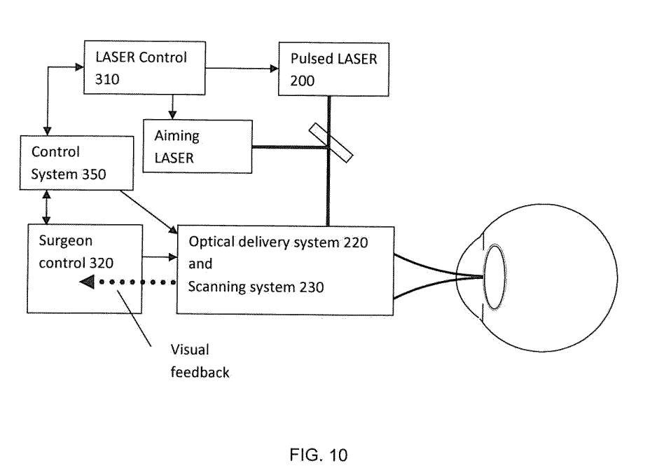

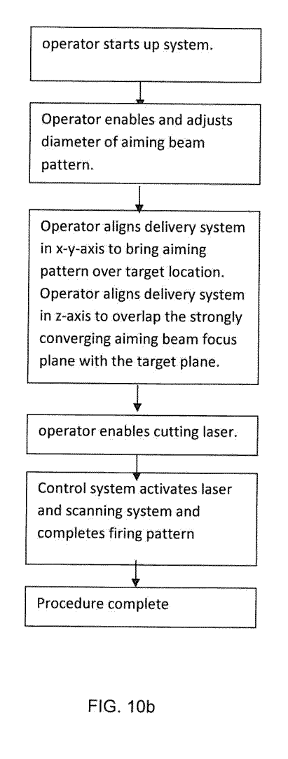

FIG. 10 illustrates a block diagram of a manual aligned system. The operator (surgeon) 320 manually aligns the delivery system 220 relative to the eye using various aiming beam patterns ether by directly moving parts of the delivery system or by controlling motorized actuators. In particular the operator aligns the lateral position and centration of the desired cutting circle with the help of aiming beam patterns.

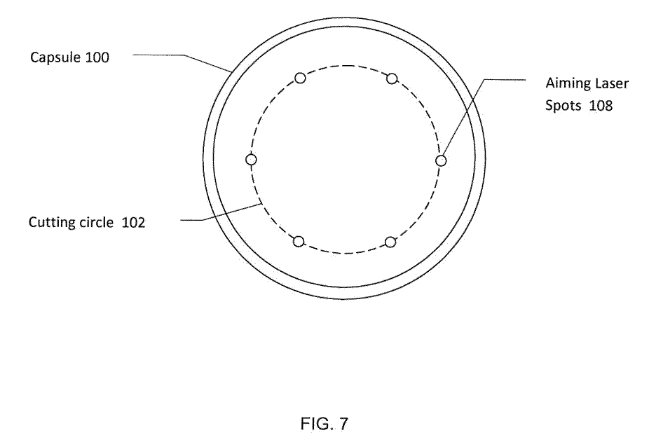

FIG. 7 illustrates such aiming beam pattern example consisting here of 6 visible laser spots 108 that outline the cutting circle 102. Patterns with more or less laser spots would be used in the same way. In the manual system, the operator centers or positions those aiming spots laterally relative to the iris or other feature of the eye and adjusts the representative diameter of the desired cutting circle.

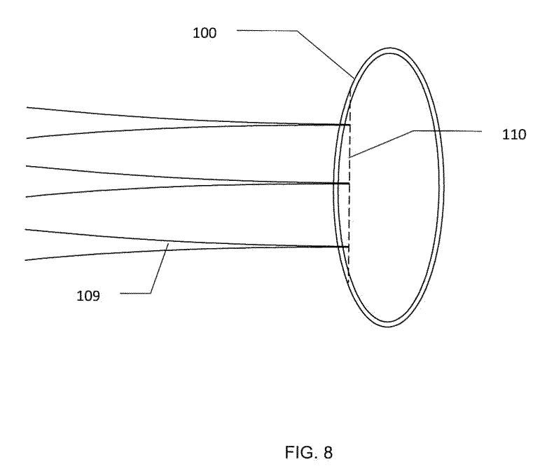

FIG. 8 illustrates a side view of the aiming beams shown in FIG. 7. Each aiming laser beam is converging to a common focal plane. The spot sizes 108 would typically be designed to be between 10 microns and 500 microns in diameter. The aiming beams are partially reflected back into the visual system (microscope/slit-lamp or imaging device) at each interface in the eye. In particular there are two very close reflections created at the interface from the anterior chamber (aqueous humor) to the anterior capsule and then around 20 microns deeper from the capsule to the lens body. Those 2 reflections combined are used to guide the delivery system alignment.

The depth alignment (z-axis) is performed by overlapping the focal plane of the aiming beams (spots) to the desired target plane on the capsule.

The goal to align the focal plane of the aiming beams to the same depth as the target plane is easy achievable by minimizing the reflections (from the target plane of the capsule) of the aiming beam through moving the delivery system back and forward (z-axis).

The focal plane of the aiming beam patterns is calibrated within the visual system of the delivery system to fall together with the visual focal plane. This further helps to make the depth alignment an easy process since the desired depth alignment will also produce the most sharp visual picture of the target plane and all other reflections of the aiming beam from other interfaces such as the cornea, will not just have a larger and therefore less intense aiming beam diameter, but will also be visually out of focus and therefore mostly not be visible at all.

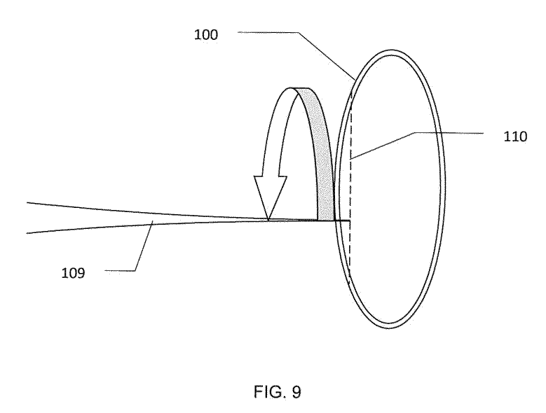

Another usable aiming beam pattern can be achieved by scanning one laser beam 109 along the desired cutting circle/ellipse as illustrated in FIG. 9. The alignment process is performed almost identical, except for the depth alignment were instead of minimizing individual spots now the circular line width 110 is being minimized. This method provides the additional advantage of detecting and correcting a possible tilt misalignment between the target plane and the aiming beam pattern focal plane. Any tilt misalignment would be noticeable by a non-uniform line thickness along the aiming beam circle. Tilt adjustments to minimize tilt can then be performed by minimizing this non-uniformity.

Once the delivery system is aligned to the target area of the eye, the surgeon enables the laser firing sequence, for example by activating a footswitch button. During this sequence a control system adjusts the scanners and optics of the delivery system automatically to complete the entire firing sequence and deliver the laser pulses to the desired target area. The operator does nothing during this phase and until the procedure is completed within a typical time of <1 s.

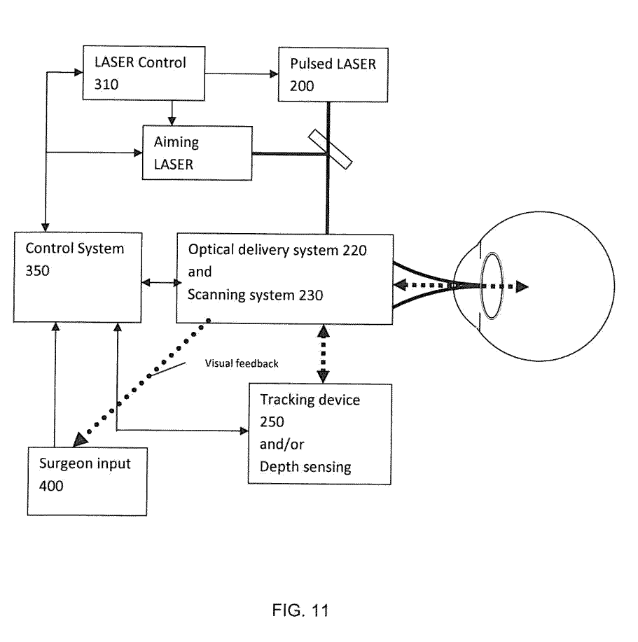

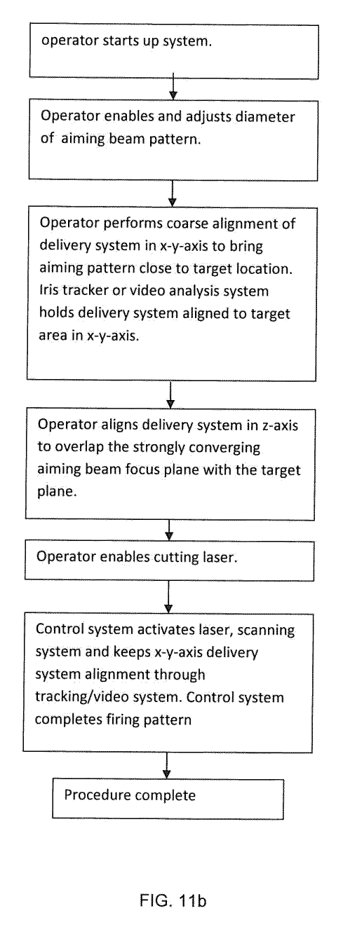

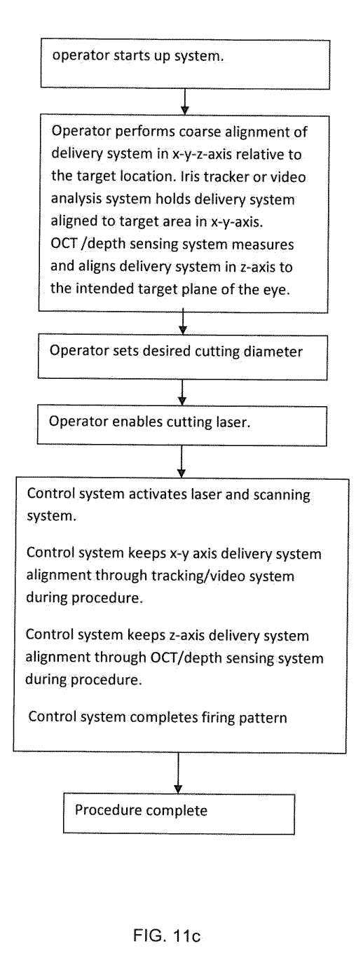

The visual feedback illustrated in FIG. 10 and FIG. 11 can be achieved through a direct microscopic view or through a camera based visual system that provides an image/video on a monitor. The optical elements of the visual feedback system might be partially shared with the optical elements of the laser delivery system.

FIG. 10b illustrates a typical flow process of the manual adjusted procedure.

Further precision of the laser cutting can be achieved by automating the alignment of the delivery system to the eye and adding continuous tracking.

FIG. 11 illustrates a block diagram of a semi-automatic (x-y-axis is automatic) and a full automatic (including z-axis) aligned system. In the semi-automatic system, the operator 400 only coarsely aligns the delivery system relative to the eye. The lateral alignment (x-y-axis) is then precision aligned with the help of a tracking system 250 such as an iris tracker (Online pachymetry, advanced eye-tracking improve LASIK. Ophthalmology Times; Vol 32, No 14, Jul. 15, 2007 p. 26.), another method for the lateral alignment would be a video analyzing system that follows and adjusts the aiming beam pattern to the desired location. The depth alignment (z-axis) would still be performed as described in the manual system.

In the fully automated system the precision depth alignment will also be measured and corrected automatically. One implementation of a depth scanner would use a optical coherence tomography (OCT) system, that provides high resolution images that contain depth information of the capsule and lens (Kurtz et al., US Patent Application: Pub. No.: US 2009/0171327). Through such a system the z-axis distance to the desired cutting plane can be measured and transmitted to a control system that then adjusts that distance through actuators inside the delivery system. The OCT system preferably is optimized to achieve a fast scanning image refresh rate so that the residual eye movement during one image scan can be neglected. One way to calibrate the z-axis of the OCT image to the z-axis of the laser focal plane of the delivery system could be done as follows: The system fires some laser pulses at a low rate into the space between the capsule and the cornea inside the aqueous humor. Those laser shots create a small cavitation bubble, that is visible in the OCT scans and therefore can be measured in distance relative to the target area of the eye, that is also visible in the OCT image.

Another system uses Scheimpflug imaging (C. Verges, “Applications of PENTACAM in Anterior Segment Analysis”, Highlights of Ophthalmology, Volume 35, No 3).

Another system to achieve automatic depth sensing and alignment of the delivery system is introduced here and uses a visual video stream from the focal plane of the microscope that also falls together with the focal plane of the aiming beam pattern. A video system including a computer picture analysis can measure and minimize the line thickness of an aiming beam pattern such as illustrated in FIG. 9 by moving the delivery system back and forward. This allows the system to stay focused on the desired target plane of the eye.

Any one of the here described preferred automated systems consist of a sensing and measurement device that transmits its data to a control system that then controls the precision alignment of the delivery system relative to the target area of the eye.

This sensing and alignment can either be performed upon operator request, for example once right after the enabling request for the cutting laser has been issued and just before the laser starts firing. This would create a one time last moment delivery system alignment correction before the firing sequence. Any remaining eye movement during the short firing sequence would not be corrected anymore.

In another implementation the sensing and alignment system works continuously before and during the firing sequence and therefore further improving the cutting precision.

In a semi or fully-automated system the cutting cylinder depth can easy be reduced from +/−1 mm so that the actual cutting time is further reduced.

FIG. 11b illustrates a typical flow process of a semi-automatic continuously adjusted procedure while FIG. 11c illustrates the fully automatic procedure flow.

Various modifications and variations of the here presented embodiments can be made by a person of ordinary skill in the art. Other embodiments of the present invention will be apparent to those skilled in the art from the present consideration. It is intended that the present specification and examples be considered as exemplary only.

1. A method for making an incision in a capsule of the fens by applying a rapid sequence of photodisruptive laser pulses into a portion of the determined target region without a contacting interface between the laser system and the eye.

2. A method of claim 1, wherein the laser pulses are applied in successive circular patterns starting posterior to the target plane of the capsule and scanning through the capsule and into the anterior region of the target plane, wherein each circular incision pattern is created by a sequence of laser pulses placed next to each other to form the pattern.

3. A method of claim 2 wherein the circular patterns are planar circles that are vertically stacked to form a cutting cylinder.

4. A method of claim 2 wherein the circular patterns is a continuous upward spiral that forms a cutting cylinder.

5. A method of claim 1 wherein the laser is delivered through an optical delivery system, that allows the laser pulses to be scanned in the desired patterns.

6. A method of claim 1 wherein the laser is aimed to the target area of the capsule of the eye using a converging visible low power aiming laser beam pattern that is being focused onto the desired target plane in the eye.

7. A method of claim 6 wherein the treatment laser starts the cutting circles posterior to the desired target plane, then progresses through the target plane and ends anterior to the target plane.

8. A method of claim 6 wherein the aiming laser beam pattern comprises: a focused aiming beam that is scanned into a visible alignment circle and its focus plane being overlapped onto the desired target plane in the eye.

9. A method of claim 6 wherein the aiming laser beam pattern comprises: multiple laser beam spots that are being focused onto the desired target plane in the eye and being arranged into a visible pattern outlining the treatment circle.

10. A method of claim 6 wherein the aiming laser beam pattern is laterally centered and is focused onto the desired target plane by manually adjusting at least some part of the delivery system relative to the eye.

11. A method of claim 6 wherein the aiming laser beam pattern is laterally centered and is focused onto the desired target plane by adjusting at least some part of the delivery system relative to the eye using electrically powered actuators and being controlled by the surgeon.

12. A method of claim 6 wherein the aiming laser beam pattern is laterally centered and is focused onto the desired target plane by adjusting at least one part of the delivery system relative to the eye using electrically powered actuators and being controlled by an automatic tracking system.

13. A method of claim 2 wherein the treatment laser circles are automatically centered in alignment using an lateral tracking device in the x-y plane.

14. A method of claim 2 wherein the treatment laser target plane is automatically adjusted using a depth sensing device.

15. A method of claim 12 wherein the automatic tracking system uses video analysis of the aiming beam pattern.

16. A method of claim 1 wherein the laser engine provides a high repetition rate of pulses so that the entire sequence of photodisruptive laser pulses is applied to the target area in less than 3 s.

17. A method of claim 5 wherein the delivery system is part of a slit lamp setup.

18. A method of claim 5 wherein the delivery system is part of a microscope that provides images of the target area of the eye to the surgeon.

19. A method of claim 1, wherein the laser pulses have a pulse duration of less than 10 picoseconds.

20. An eye-surgical device, comprising: a pulsed laser, configured: to be directed in a rapid pulse sequence at a capsule of an eye to perform an incision of the capsule without a contacting interface between the laser system and the eye.
